(19) United States
(12) Patent Application Publication
(10) Pub. No.: US 2023/0233099 A1
(43) Pub. Date: Jul. 27, 2023

(54) BREATH GUIDE DEVICE AND METHOD

(71) Applicant: Breathpen Limited, London (GB)

(72) Inventors: Ben Mather, London (GB); Alex McKemey, London (GB)

(73) Assignee: Breathpen Limited, London (GB)

(21) Appl. No.: 17/999,461

(22) PCT Filed: May 21, 2021
(86) PCT No.: PCT/EP2021/063729

§ 371 Date: Nov. 21, 2022

(30) Foreign Application Priority Data
May 22, 2020 (GB) 2007715.2

(51) Int. Cl. A61B 5/087; A63B 23/18

(52) U.S. Cl. CPC A61B 5/087; A63B 23/18

(57) ABSTRACT

An apparatus and methods enhances the energy levels of a person, in particular the relating to guiding a person's breathing to enhance energy and health. The device is a breath guide device having an airway, breathflow guiding equipment, a control unit capable of communicating with a memory unit. The control unit is arranged to control the breathflow guiding equipment in dependence on data held in the memory unit. A mouthpiece suitable for placement at a user's mouth may be included. The breathflow guiding equipment includes an electronically operated valve for regulating the flow of breath through the airway.

### FIELD OF THE INVENTION

The invention relates to apparatus and methods for enhancing the energy levels of a person. In particular the invention relates to guiding a person's breathing to enhance energy and health.

### BACKGROUND OF THE INVENTION

Conscious breathing activates the body's relaxation response, which in turn reduces blood pressure, which in turn lowers the risk of stroke and improves cardiovascular health. It is also good for digestion and general immunity, both of which are impaired by stress. Conscious breathing can also provide relief from negative emotions, heal past traumas, and accelerate self-improvement,

Breathing is one of the few bodily processes that can either be voluntary or involuntary. Breathing can take place automatically without thinking about it, or can be altered consciously and at will. Because of this unique relationship between thinking and bodily processes, breathing pattern scan play a deciding role in the affect of stress.

Breathing is one of the most sensitive indicators or warning signs of stress, because it is a vital link between minds and body. By consciously increasing awareness of breathing and by practising breathing exercises it is possible to improve well-being, decrease stress and help to bring about interconnectedness between mind and bodies. Also conscious breathing exercises increase the level of energy in a person's body.

The Textbook Of Medical Physiology notes that all chronic pain, suffering, and disease are caused by a lack of oxygen at the cell level.

The human body is comprised of a structure, animated by the energy we get from breathing. This energy sustains, heals and renews the mind and body. It is known in cultures throughout the world by various names, such as Chi or Prana. This energy will be referred to as prana in the description below. Prana and electrons have overlapping properties in the subtle and gross domains respectively. Electrons can reduce free radicals (inflammatory dis-ease forming) and come from naturally available earth elements—including air (the breath), grounded-earth, water and fire. Water self-orders, and can ‘buffer’ between electron and proton flow leading to ‘proticity’ capabilities (creating a battery power within the body).

Prana is now starting to be measured: its interaction with the biosystem has been the subject of study in Ayurveda and Yoga. Energetic Medicine and Biophysical integrative studies are starting to provide further insight. Even the definition of death is based on loss of prana. Chi, the equivalent of prana in Traditional Chinese Medicine (TCM) is claimed to be measured through instruments and its loss has been observed at the time of death. Electrons are also being studied more closely now, especially their conductive, antioxidant and inflammatory-affecting capacities within the health of individuals (discussed in later chapters). Here, we have a complex and newly explored area of subtle energies that impact health.

From the perspective of the philosophy of Yoga, which is directed towards the realisation of our essential nature, prana is the primordial energy of the universe. Prana is dispersed through the entire material world. ‘Prana is both macrocosmic and microcosmic and is the substratum of all life: Mahaprana (the great prana) is the cosmic, universal, all-encompassing energy out of which we draw substance through the “breathing process”.’

This ‘duality’ aspect of life is constantly being reminded to all of us. This world of polarities has been symbolically represented in yoga as the pingala (carries feminine, lunar energy beginning in muladhara and ending in the left nostril) and ida (carries male, solar energy beginning in the muladhara and ending in the right nostril). Other philosophical forms of this ‘duality’ include the Yin and Yang depiction, as in Taoism. These symbols, by their visual representation, help to understand and relate to the universal laws that exist: those of opposites and polarities.

According to the yoga shastras, this duality is the underlying energy structure which gives the body life. Scriptural texts cite 72,000 nadis or flows of energy throughout the human body.

The Nadis (or ‘tubes’) are the channels through which, in traditional Indian medicine and spiritual knowledge, the energies such as prana of the physical body, the subtle body and the causal body, are said to flow. Within this philosophical framework, the nadis are said to connect at special points of intensity, the concentrated energetic centres of the body, the ‘chakras’ ('wheels' or ‘disks’) of spinning light-energy.

These flows have various interpenetrating and interdependent levels: just as sound has harmonics and subharmonics. They may be physical (nervous, circulatory, lymphatic), pranic (more subtle energies still relating to the body, like metabolism), mental (through currents, frequencies or electromagnetic (EM) wave resonance) or psychic (dreams or visions).

Duality of energy flows throughout the body via channels of the ‘Pingala’ nadi which conducts prana shakti ((body) vital energy), ‘Ida’ nadi which conducts manas shakti ((mind) mental energy), existing as twin energy flows. The number of possible twin energy attributes for ida and pingala are endless: e.g. left nostril/right nostril, introversion/extroversion, negative/positive, female/male, etc. This duality property can be seen in all of nature. Translated into the human body this can also be applied: both the macrocosm and microcosm are based on these two dual polarities: arteries/veins; PNS sympathetic (fight and flight response)/PNS parasympathetic (rest and digest response); CNS sodium/potassium exchange pump, etc . . .

The question of ‘flow’ extends further into the realisation of pressure or energetic wave forms. This relativity, in relation to the function of the breath, will be discussed further in later chapters, as part of the Olfactory process and forms an important explanation behind the benefits of the most ancient alternate nostril breathing exercise.

When dual aspects of ida and pingala merge (balance), the two nadis partner together and cross over the spine, meeting in the center of the sushumna nadi: a third force comes into existence. This force is the flow of spiritual energy in the sushumna nadi.

Sushumna is a Sanskrit word meaning “very gracious” or “kind” and is also the name for the central nadi in the body. The sushumna runs down the central axis of the body, through the spinal cord. The sushumna nadi connects the muladhara chakra (root or base chakra located anatomically at the pelvic plexus, perineum) to the sahasrara chakra (crown chakra located anatomically at the very top of the skull), and is the path for the ascent of kundalini energy up the base of the spine, through the chakra bodies and into the sahasrara (crown of the head). It is considered the central channel for the flow of prana throughout the body, and unites all other chakras in the body.

By being aware of the sushumna and how it affects the flow of prana throughout the body, practicing yogis can concentrate on awakening and increased spiritual energy flow, distributing energy throughout the chakras. Chakra meditation and breath awareness practices, breathing exercises and conscious connected breathing sessions are used to stimulate the sushumna from its resting state. Hence, the important ascension of Kriya Yoga**.

*In his book, Breaking the Death Habit: The Science of Everlasting Life, Leonard Orr (1998), suggests that this free-flowing of energy starts with the free flowing of the breath and the release of the breathing mechanism. As seen in the breath of newborns and infants, the connected breath can be mimicked by the learned phenomena of uninterrupted coupling of the inhale with the exhale and relative exercises can be carried-out to improve the access to this pranic flow, ultimately accessing the sushumna nadi.

Paradoxically, mastering the breath (starting with the basic performance and mastering of basic breath exercises) enables and engages the benefits of the breath. It is important to understand how the conscious prana energy/pranayama affects the mental and emotional mind and energetic body, and all the other areas in life mastery it enables. It is thus beneficial to encourage an undertaking of conscious breathing as a valid and qualified healing modality. It is known to use controlled slow-breathing techniques as a means of optimising physiological parameters that appear to be associated with health and longevity, and that may extend to disease states.

**The development of ‘Breath Exercises’ forms the basis and background for Kriya Yoga (action/deed effort with connection/unity): dispensing the ‘power of the breath’ in a tight set of disciplinary practices for the purposes of protecting people from the dramatic physical and emotional changes that this ‘power’ produces in human personalities, as it purifies and heals the emotional and energetic mind and body. Breathing techniques and patterns, historically, have been regularly advocated for relaxation, stress management, control of psycho-physiological states and to improve organ function (Ritz and Roth, 2003). There is a need for the advantageous facilitation and manipulation of breathing techniques.

The conscious connected breath has the ability to dramatically improve health, happiness and success. The conscious connected and facilitated breath has multiple roles and is featured in the healing of asthma, migraines, epilepsy, the common cold, chronic pain, fatigue and associated mental disorders including: depression, anxiety, insomnia and stress. Inflammation causes most dis-ease. Anatomically, there is a favorable equilibrium (balance in breathing pressures) with breathing, which can be easily disrupted by fatigue or prolonged sympathetic (excitatory) nervous system arousal as seen with stress, or other prolonged negative emotional states. The therapeutic goal of yoga is that it can reduce or alleviate some of the chronic negative effects of stress. This stress relief is one reason that breathing (pranayama) is central to yoga (Kriya) practices.

It is therefore an object of the invention to provide a device and method for implementing breathing exercises.

### SUMMARY OF THE INVENTION

In an embodiment of the invention, a breath guide device is provided having; an electronically operated valve for regulating the flow of breath through the airway, the guiding equipment further comprising haptic feedback apparatus including a vibration unit, the device further having a control unit capable of communicating with a memory unit, the memory unit capable of storing data representing an ideal breath flow rate, the device further including a breathflow measurement unit in the airway arranged to measure the breath flow rate of a user and wherein the control unit is further arranged to compare the data representing the ideal breath flow rate with the data representing the measured breath flow rate of the user, and to control the valve and the vibration unit in dependence on the result of the comparison. This device is a basic unit, which is capable of delivering a breathing exercise stored in the memory. The controller instigates a controlling step with an output corresponding to instructions for a breathing exercise.

A mouthpiece suitable for placement at a user's mouth may be included, wherein the breathflow guiding equipment includes an electronically operated valve for regulating the flow of breath through the airway. The valve opens and closes depending on the breathing exercise that is selected from the memory.

The breath guide device could be just a nasal piece comprising two nasal conduits for engagement with a user's nostrils and wherein the breathflow guiding equipment includes an electronically operated valve in each of the two nasal conduits for regulating the flow of breath through the airway. The guide could also comprise a combination of the nasal piece and mouthpiece.

The data capable of being held in the memory may be data defining a breathing exercise.

If the breath guide is a mouthpiece only, it may be arranged to close the valve in the mouthpiece if the user's breath flow rate is detected as being higher than the preset breath rate.

If the breath guide is a nasal piece only, then the breath guide device may be arranged to close one of the valves if the user's breath flow rate is detected as being higher than the preset breath rate.

The breathflow guiding equipment may include a plurality of visual indicators, and/or an audible indicator, which may be capable of reproducing pre-recorded spoken words. The audible indicator may be arranged to operate if the user's breath flow rate is detected as being higher or lower than the expected breath flow rate.

The breathflow guiding equipment may include a vibrational indicator, arranged to operate if the user's breath flow rate is detected as being higher or lower than the expected breath flow rate.

The memory unit of the breath guide device may be located within the device or may be arranged remotely by means of a remote communication system.

The breathflow measurement unit may include one or more IR sensors, and/or one or more LiDAR sensors and/or it may be one or more turbine sensors.

In a further embodiment, a method of operating a breath guide device is provided, including the steps of reading data representing a breathing exercise held in a memory unit and controlling breathflow guiding equipment located in the device in accordance with the data representing a breathing exercise.

The breathflow guiding equipment may be at least one valve and the method may involve opening and closing the at least one valve in accordance with the data representing a breathing exercise.

The breathflow guiding equipment may be a plurality of indicator lights and the method involve illuminating the indicator lights in accordance with the data representing a breathing exercise.

The breathflow guiding equipment may be a sound emitting device and the method involve emitting a sound in accordance with the data representing a breathing exercise.

The breathflow guiding equipment may be a vibration unit and the method involve causing the vibration unit to vibrate in accordance with the data representing a breathing exercise.

The method may further include the steps of measuring the breathflow of a user flowing through the device, comparing the measured breathflow with a value representing an expected breathflow in accordance with a breathing exercise and further controlling the breathflow guiding equipment in dependence on the result of the comparison, to guide the user's breathing into alignment with the breathing exercise.

The method may further include a calibration stage, comprising the steps of instructing a user to breathe all the way out to empty their lungs, instructing a user to breathe all the way in to fill their lungs, measuring the total breath flow as the user fills their lungs, measuring the time it takes for the user to fill their lungs, calculating a constant representing a characteristic of the user's breathing behaviour.

The method may further include the step of using the constant for the comparison of the breathflow with the value representing expected breathflow.

### BRIEF DESCRIPTION OF THE DRAWINGS

FIG. 1 is a perspective view of a breath guide device in accordance with an embodiment, for use in delivering breathing exercises for the mouth only.

FIG. 2 is an exploded perspective view of the breath guide of FIG. 1.

FIG. 3a is circuit diagram showing the arrangement of the electronic elements of the device of FIG. 1.

FIG. 3b is a schematic diagram showing an arrangement of indicator lights of the device of FIG. 1.

FIG. 4 is a perspective view of a breath guide device in accordance with an embodiment, for use in delivering breathing exercises for the nostrils only.

FIG. 5 is a perspective view of a breath guide device in accordance with an embodiment, for use in delivering breathing exercises for a combination of the mouth and nostrils.

FIG. 6a is a perspective view of a breath guide device in accordance with an embodiment, for use in delivering breathing exercises for a combination of the mouth and nostrils, where a combination oral unit is shown detached from a base unit.

FIG. 6b is a perspective view of the breath guide of FIG. 6a, where the oral contact unit is shown attached to a base unit.

FIG. 7 is a perspective view of an embodiment of a breath guide which is connected to a smart phone.

FIG. 8 is a perspective view of an embodiment.

FIG. 9 is a perspective view of a hands-free embodiment.

### DETAILED DESCRIPTION OF THE PREFERRED EMBODIMENT

Conscious breathing exercises benefit the individual by: creating awareness and calming; reducing chronic pain; purifying the blood; resetting the nervous system and response; stilling or quieting of the mind; creating a grounded sense of stability and heart-centred existence; creating a sense of balance and harmony; increasing memory recall; gaining emotional and physical control; reducing stress and giving a feeling of relief; increasing the depth of breathing and the capacity of the entire breathing mechanism; increasing physical and mental energy; feelings of alertness and reinvigoration; creating peaceful, pleasant and often euphoric sensations.

The breath exercises, if performed daily for a continual cycle of a 90 day period (missing a day returns to the beginning of the 90 day cycle) will fundamentally alter life and health positively—incorporating awakened being with the engagement of sushumna nadi and kundalini ‘life-force’ energy.

The exercises, especially if performed over an extended period, will have considerable and important physiological and cellular changes: awakening and uncovering many deep subconscious emotional ‘blocks’—stored in a dysfunctional breathing mechanism and released as the breathing mechanism improves.

It is important that these blocks, traumas, stored emotional memories and dysfunctional breathing patterns are released and worked-through. This is where the deeper healing then occurs and physiological and cellular changes can occur. Electromagnetic wave resonance (observed through EEG readings—Heartmath) will slow from Beta to more sustained Alpha, and often Theta low frequency/lucid resonance, and the external and major internal benefits that these types of resonance provide. There also exists an opportunity to reflect personally on ‘shifts’ or changes: in collaboration or compassionately seeking assurance with others who are going through similar responses from the exercises and in the process also creating this type of environment, supporting positive health-change: individually and collectively.

Regular Tidal (inhalation and exhalation during restful breathing) and Hyperpnea (deep breaths with no change in respiratory rate) slow-breathing of gently ‘pulling’ on the inhale and merging and connecting with a natural ‘unforced’ release of the exhale has enhanced autonomic, cerebral and psychological flexibility in a scenario of mutual interactions: inclusive of voluntary regulation of internal bodily states (enteroception), and the other is associated to the role of mechanoceptors within the nasal vault in translating slow breathing in a modulation of olfactory bulb activity, which in turn tunes the activity of the entire cortical mantle. Overall increased emotional control and psychological, if progressed, usually in senses of well-being results. Anatomically, the Diaphragm has direct and indirect influences on emotional and psychological pathways. Its facilitated manipulation and modulation can thus be greatly beneficial.

Deep prolonged inspiration results in prolonged contraction of the diaphragm, This, in turn, will increase the expansion of the chest cavity, Coupled with occlusion of the Gastropharyngeal Junction/Glottis opening, results in decreased Intrathoracic Pressure allowing air to regularly flow and enter and expand the lungs and musculoskeletal housing.

Relaxed expiration, allowing the air to flow naturally, allows the diaphragm to return, unforced, to its original shape. The remaining components of the breathing mechanism, naturally contract to resting state.

The consistency of connection, without disruption of the inhale, connected immediately to a natural exhaled release demonstrates a biofeedback* that achieves a steady increase in Heart rate variability (HRV) patterns proposed to reflect physiological “coherence” and entrainment of heart rhythm oscillations to other oscillating body systems.

Dysfunctional breathing patterns are associated with decreased ability to achieve healthy increased HRV patterns that reflect cardiorespiratory efficiency and autonomic nervous system balance. This suggests that dysfunctional breathing patterns are not only biomechanically inefficient but also reflect decreased physiological resilience.

*Biofeedback is the process of gaining greater awareness of many physiological functions of one's own body, through mind-body techniques implicating the use of feedback, mostly visual and auditory, to gain control over involuntary bodily functions: gaining voluntary control over such things as heart rate, muscle tension, blood flow, pain perception and blood pressure. Availability is, at present, commercially viable by using electronic or other instruments.

### Effect of Breathing Exercises on Cardiovascular System (CVS):

A few studies also reveal that biofeedback using respiratory control, relaxation and meditation techniques can increase HRV. HRV seems to be a far better indicator of parasympathetic activity and interplay than Heart Rate (HR) alone. For now, there is insufficient data to determine if paced respiration or subjective relaxation is necessary or sufficient for the efficacy of HRV biofeedback.

Every controlled breath in, will increase HR in order to speed the flow of oxygenated blood around the body. Expiration allows HR to decrease. The variability (HRV) is regulated by the cardiac inputs of the vagus nerve (VN). On inspiration, VN is suppressed, on expiration it is activated: thus the greater HRV (on inspiration and expiration) the greater the cardiac Vagal Tone (VT). Although the literature is modest, reviews suggest that the use of biofeedback with relaxation, deep tidal breathing and meditation approaches result in increased HRV and parasympathetic activity. Slow-paced control of diaphragmatic inspiration and long-slow ‘unforced’ relaxed expiration directly increases HRV and allows a person to control a key element of the autonomic nervous system and the sympathetic/parasympathetic autonomic innervation.

In contrast, a reduced HRV seems indicative of decreased cardiac VT and elevated sympathetic activity seen in anxious and depressive patients and reflects a deficit in flexibility of emotional response. Further evidence of links between parasympathetic activity and increased HRV and LF power (Low Frequency power (LF): frequency activity in the 0.04-0.15 Hz range LF/HF Ratio: A ratio of Low Frequency to High Frequency is considered being indicative of Sympathetic to Parasympathetic autonomic balance.

LF power seems to provide an index not of cardiac sympathetic tone but of baro(receptor)reflex function physiological response mechanisms. An Increased HRV and LF power therefore results in reduced haemodynamic effects: lowered pulse rate and decreased blood pressure.

A decreased arterial blood pressure will result in an increased blood flow. This is modulated via depressor and cardioinhibitory feedback that initiates the baro(receptor)reflex, resulting in a reduced VT and increased HRV. This elicits the parasympathetic pathways again and an individual's outward experience of a calm and restful state.

### Effect of Breathing Exercises on Gaseous, Ionic and Metabolism:

Slow, steady contraction of the diaphragm during inspiration results in changes in chemical rebalancing of the CO2 levels in the blood and return to alkalinity (mild, measured and balanced levels for more optimum physiological and metabolic functioning). Physiologic measurements of respiration at follow-up show that patients can learn to alter their Carbon Dioxide Pressure (P(CO₂)) levels and respiration rates as they notice and feel the repeated benefits they have been taught in exercises.

### CNS/PNS: (Central & Peripheral Nervous Systems—Sympathetic/Parasympathetic Activity):

During slow inspiration, stretching of the lung tissue produces inhibitory signals by action of slowly adapting stretch receptors (SARs) and hyperpolarization current by action of lung fibroblasts. Both inhibitory impulses and hyperpolarization current are known to synchronize neural elements leading to the modulation of the nervous system discussed by way of the vagus nerve (Cranial nerve (CN) X) and decreased metabolic activity indicative of the parasympathetic state (rest and digest).

Deactivation (by slow connected breathing) results in a simulated ‘more asleep’ feeling with decreased phrenic n innervation.

### Anti-inflammatory Activation:

Persistent and decreased low vagal tone, however, has been associated with chronic inflammation. In individuals' with chronic pain and clear inflammation, there will most likely be an extremely decreased vagal tone: this can be readdressed and corrected, with time and patience, by utilising monulated conscious breathing exercise patterns.

### Cerebral & Limbic:

With slow-controlled breathing there appears to be increased cerebral effects of volitional control and attentional modulation occurring as a result of various neural heart, lung and limbic cortex hyperpolarisation, whose effects originate at the brainstem in the medulla oblongata. In particular, the effects of decreased phrenic activity in the medullary Per-Botzinger Complex with associated sympathetic innervations, and Increased vagal n activity in the Crural Area, resulting in an increased parasympathetic activation.

The Amygdala shifts to open up feelings, going past entrenched emotions. The amygdala helps coordinate responses to environmental shifts, especially those that trigger an emotional response. This structure is seen to have an important role in fear and anger. The amygdala ‘hijack’ can be eased or stopped by consciously activating the frontal and prefrontal cortices, the rational, (conscious), logical part of your brain. Conscious practice and persistence of this hijacking can exist through engaging meditative states and modulating breathing patterns, with initial actions/steps in acknowledging (gaining awareness) that al threatened or stressed emotion or feeling is present and that the sympathetic fight-or-flight response has been activated. Conscious connected breathing with this ‘awareness’ or feeling exercise pattern aids this awareness and provides an opportunity for the override function to occur.

### Pranic Description:

Prana (air) is pulled from the root, on slow inspiration, (In—Spirit (eternal abundant pranic energy)) and up through the crown and out. The steady slow ‘pull’ allows the Prana energy to dissipate throughout the body, through all the nadi channels with active attention and awareness placed to the intentional ‘feeling’ of the flow of breath around the body—cellular bathing of prana effects occur with intention. The relaxed exhale (Ex—out/from hale—wave/salute), with the coupled intention aims at removing any blocks, toxins or unwanted energies.

The consistent pressure wave generated by the breath awareness and other exercises could have seen the subsequent reduced cycles of electromagnetic signals (increased Alpha) which have ‘informative’ effects through extracellular and intracellular matrices (analogous system) translated through piezo (pressure) energy.

The living (analogues) matrix (composed of the proteins of connective tissue/collagen fibres) extends all the way through the body, even down into the centre of the cells and the nucleus. The connective tissue is a semi-conductive analogous system, so it can move electrons and the piezo-electric effect of pressure has a generated electrical current force, under the right circumstances. Our consciousness, and its attention is limited: there is other information being gathered all the time, potentially subconscious uptake.

Analogous theories extend further to support the extra/intracellular interplay further through microtubular-assisted-proteins running intertwined and dynamically into cells: this piezo-electric effect and variations in flow and conductance could therefore have effects on the living plasmonic (liquid) crystalline

matrix/RNA/DNA/myosin/keratin/elastin and genes (epigenetics).

The ‘digital’ nervous system does not do this and is different (working by emitting, or not, with slower electrical currents and neurotransmitters). Information from wave energy is potentially split into digital (neurological) as explained prior and/or into the connective-tissue system ‘analogous’ (which places its theoretical attention on energy medicine crystalline matrix analogy). Due to the matrix being ‘spiral’ it has the ability to generate conductivity through piezo-electric effects, which are constantly occurring extracellularly and intracellularly. These matrices receive packages of information in pulsed (PEMF) or coded form information (David Bohm) (like the grooves on an old LP record and the stylo-reader).

1. Breathing also generates varying EM patterns and could therefore potentially alter ‘protocity’ (Hydrogen proton availability due to electronic changes of matrix function (cellular charge)). The ‘digital’ nervous system does not do this and is different. Information from the EM waves is split into digital (neurological) as explained prior, and/or into analogues (energy medicine interpretation-crystalline-matrix analogy).
Emotional memories (information wavelengths) store inside the living matrix and DNA. Epigenetics is a plausible and realistic theory. These memories stored can be potentially altered by shifting EM fields and different wave patterns (possibly influenced by the breath which also remains to be seen).
The discovery of ‘Intergens’—protein blocks (collagen) which has connections between that of extracellular to intracellular matrix—thus DNA inside—epigenetics—(epi (above) so from the environment and the genes)—thus not so much genetic expression itself though more a ‘dualistic backwards and forward information system that is occurring. (progressing from Crick theories (Watson and Crick) and one way cause and effect ideology).
EM signals—form and grow extending microtubules (tubulins) through the matrix from extracellular piezo energy, which, in turn. Encodes then a digital play—the encoded information through microtubule-assisted-proteins ventures into Nuclei of the cells and DNA encoding structures (the hard-wiring).
The (electro)magnetic changes cause de/repolymerisation of strands
*Blocked emotions and memories—molecules of emotion found in all receptors and cells.
Intracellular matrix EMagnetic fields are generated through auras and chakras—mind 60×/heart 5000×.
Magnets are unhindered—Electrical information (in the form of thought waves, or possibly breathing patterns—further studies required) has an associated magnetic resonance (Ampere's law). Faraday's magnetic field can cause conductive change too.
Signals are passed through the spiral (fibonacci—golden ratio) generating an equal magnetic field and electrical field component though will generate out through the path of least resistance. Equally, we are receiving information externally too, having impacts on the subtle nature of our biological system.
Living systems are highly non-linear, small changes can have exponentially large outcomes. ‘Coherence’ also plays a role here, using smaller and less energy with ‘pulsing’ effects of how the information is transmitted.
The HeartMath Institute provides evidence that the body is entangled with the outside environment—the heart can predict events prior to our visualization.

2. A chronic focalisation of disharmonious energies will result in Inflammation: the cause of most dis-ease. Free-radicals tend to arise in inflammatory responses causing damage to cells and vessels. Electrons can reduce free radicals and can be sourced from naturally available earth elements—including air (and potentially the pranic effects of breathing, again remains to be seen or researched)
There is a noticed reduced metabolic activity in parasympathetic states and increased HRV—therefore a reduced inflammatory response in conscious breathing states.
Analogous interpretation of inflammation prevents flow of ‘information’ and free radical generation (mopped-up by antioxidants) and locked in by the body—surplus electrons (proticity changes) can alter the inflammatory process and disrupt inflammatory pathways. We can get extra sources of electrons from pranayama/earth/nature.

The various breath guide devices described herein enable the correct and proper augmentation of the breath and its limitless possibilities. The breathing device teaches how to breathe and harness the power of breath, i.e. the ability to acquire or source the natural energy available in the environment to improve health and wellbeing. The breath has the power to heal and prevent anxiety, depression, addiction, stress etc and allows mastery of body and mind. Direct use of the device can bring calmness, relieve anxiety and stress, can help focus and concentration, bring peace of mind and de-clutter thoughts. It can replace the smoking urge.

The device is portable and can be used in any location or setting, at work or at home, in isolation or in a global group meet up. It can be used while doing other activities, for example, studying in meetings, while travelling.

Whilst there are beneficial immediate effects of the device use, sustained use over longer-periods increases and enhances health benefits.

In an embodiment, a device is provided that guides or manages a user's breathing patterns. The device is pre-loaded with breathing exercises for a user to follow and can measure a user's breathing patterns to determine whether the breathing exercises are being followed correctly. If the exercises are not being followed, this is indicated to the user so that they can take action to correct it.

The device includes various displays and controls but substantially is an intelligent duct that can be used to guide the user's breath entering or leaving their mouth, nose or both. Sensors are provided to measure the flow of breath and valves are provided to guide the user's breathing patterns, correcting them if they deviate from the exercise.

A version for use with the mouth only is described in detail below.

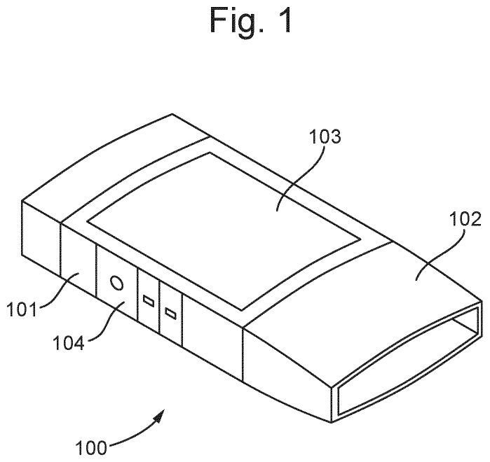

In an embodiment shown in FIG. 1, the device 100 includes a body 101 and a mouthpiece 102. The body and mouthpiece combine to provide a duct, or channel, or airway so that when the device is raised to the user's mouth, they can breathe through the device.

The mouthpiece 102 is a passive tube, which can be removed for cleaning and for replacement. Different sized mouthpieces can be used depending on user preference and comfort. A typical mouthpiece inlet would be oval in shape for comfort with an aperture of approximately 1cm high by 3 cm wide.

The body 101 of the device includes a duct to guide air to and from the mouthpiece. The body 101 includes a screen 103 for displaying information such as performance indications, instructions, feedback, menus or other data. The body 101 also includes buttons 104 for controlling the device, such as turning it on and selecting breathing exercises.

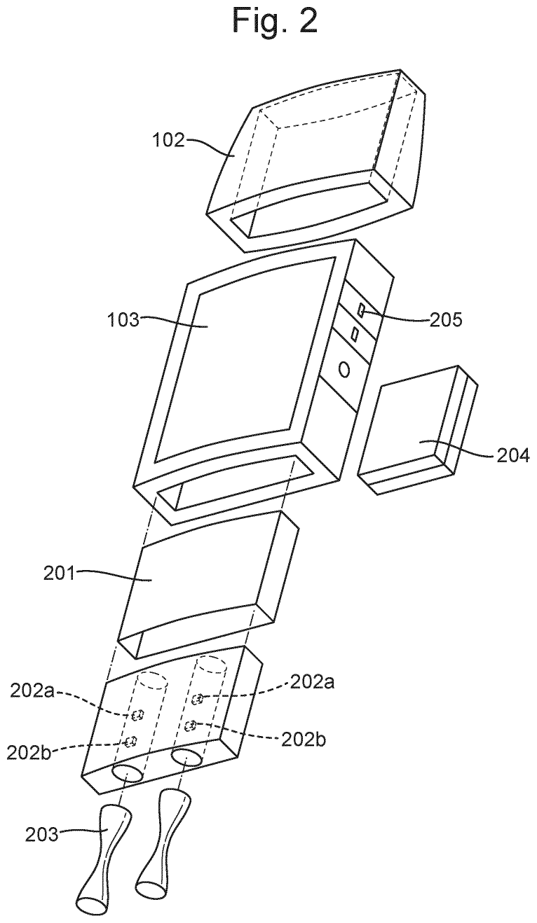

FIG. 2 shows an exploded view of the device. Within the body 101 there is a breath flow sensor unit 201.

The body also includes a power supply such as a battery 204. The body also includes a servo operated valve (not shown) for stopping or allowing breath flow. The body also includes a port 205 for charging and data transmission and reception.

The flow sensor unit 201 is arranged to detect the breath flow of a user, in particular to measure the velocity of the flowing gases as the user inhales and exhales. A number of techniques can be used for measuring the velocity of flowing gases, including turbines 203 in the gas stream connected to dynamos, thermal probes (not shown) to measure the cooling effect, Pitot tubes (not shown) to measure the pressure of the flowing gas and venturi restrictions (not shown) to create a measureable pressure drop. When a combination of turbines and dynamo are used, electromagnetic resistance can be applied to impede the flow of breath if necessary. Furthermore, the possibility exists for a user to breathe heavily into and out of the device to cause the dynamos to generate sufficient electricity to power the device for a period of time. This hyperventilating effect can have health benefits if used under controlled circumstances.

Non-contact gas velocity sensors can be used. These include infrared sensors 202a, 202b. IR sensor 202a is spaced apart from sensor 202b in the flow of breath. As exhaled breath, or inhaled air, passes the sensors, small variations in temperature are sensed. These variations can be tracked and the time it takes for them to pass from one sensor to the other is used to determine the velocity of the flow and flow direction.

FIG. 2 shows the use of both IR sensors and turbines in the same device. However, it is adequate for a device to have only one type of sensor for measuring the flow rate.

Another method of sensing the flow rate is Micro-LiDAR. LiDAR stands for Light Detection and Ranging. The velocity of the flow is measured by sending laser light into the gas flow and measuring the light scattered from the molecules in the flow. The sensors can be configured as spectroscopy, induced fluorescence and differential measurement devices to provide flow composition, gas density, temperature and pressure data.

The body 101 also includes sound producing unit such as a speaker (not shown) and a vibration-producing unit (also not shown).

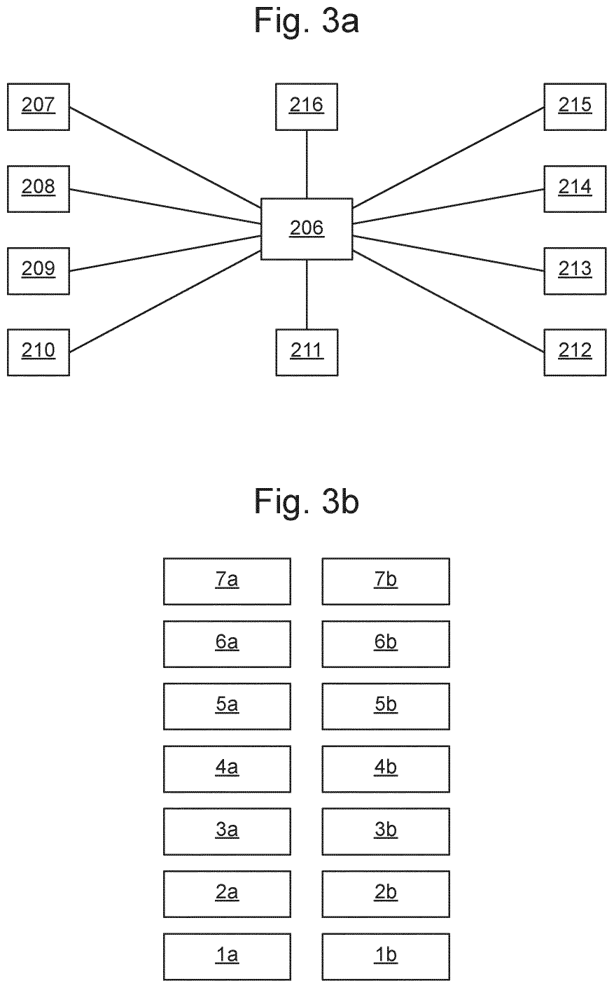

The body 101 also includes electrical control circuitry as shown in FIG. 3a. A central processing unit CPU 206 is provided. The CPU handles data flow around the device. It is connected to a memory unit 207 for storing breathing exercises and other data relating to breathing performance. The CPU and other electrical components are powered by battery 208, shown as reference 204 in FIG. 2. Flow sensors 209, shown as 202a, b and 203 in FIG. 2, are arranged to transmit readings to the CPU 206. Data and charging port 210, shown as 205 in FIG. 2, is provided to upload and download data to and from the device, such as breathing exercises and also to charge the battery 208. The CPU 206 is arranged to send valve control signals to valve servo 211. The user can interact with the device via control buttons 212, shown as reference 104 in FIG. 1. Indicator lights 213 are provided to indicate various states of the device and the user's breathing performance. The indicator lights can be LEDs mounted on the body or they can be rendered on the display. A sound module 214 is provided for audible indications of the breathing exercises. It can also indicate a user's performance in relation to the exercises. The screen 215, shown as 103 in FIGS. 1 and 2, is connected to the CPU. A wireless communication module 216 such as Bluetooth® is provided to communicate wirelessly with other devices such as a smartphone. A filter may be provided over the outlet, which may be a mesh, gauze, fabric or electrostatic type.

Indicator lights 213 are arranged in two columns of seven lights, as shown in FIG. 3b. The first column of lights 1a to 7a indicate idealised breathing in accordance with the current breathing exercise and are referred to as guide lights. The second column of lights 1b to 7b indicate the user's actual breathing as measured by the sensors 202. The indicator lights represent the energetic state of the user, where the amount of air present in a user's lungs is equated to the energy they are drawing into and up their body. When the lights are not illuminated, their breath is all the way out. When the lights are fully illuminated, their breath is all the way in, this represents utilisation of the individual's full breathing mechanism. These lights are referred to as energy level lights.

The first set of guide and energy level lights 1a, 1b represent 1/7^{th} of a full breath and energy arriving in the lowest part of the breathing mechanism (the perineum). The colour of the first lights 1a, 1b when illuminated is red.

The second set of lights 2a, 2b represent 2/7^{th} of a full breath and energy arriving at the belly. These lights are orange.

The third set of lights 3a, 3b represent 3/7^{th} of a full breath and energy arriving at the sub-sternum/diaphragm/solar plexus. These lights are yellow.

The fourth set of lights 4a, 4b represent 4/7^{th} of a full breath and energy arriving at the heart area. These lights are green.

The fifth set of lights 5a, 5b represent 5/7^{th} of a full breath and energy arriving at the throat. These lights are light blue.

The sixth set of lights 6a, 6b represent 6/7^{th} of a full breath and energy arriving at the forehead. These lights are dark blue.

The seventh set of lights 7a, 7b represent a full breath and energy arriving at the crown. These lights are indigo.

When the user first uses the device they are prompted to enter a calibration mode. The calibration mode is used to establish a value representing the lung capacity of the user. The calibration mode involves the device instructing the user to breathe all the way out and to press a button 212. When they have done so, to then breathe all the way in and press the button 212. As they breathe in, the sensors 202 measure the flow rate of the breath each millisecond and a timer measures the total duration. The average flow rate is calculated and multiplied by the total duration. The resulting figure, called the lung constant, represents the lung capacity of the user. The lung constant is used to ensure that the correct indicator lights 213 are illuminated during use to represent the degree of fullness of the user's lungs.

The calibration step is repeated periodically, for example weekly or monthly, because the breathing exercises cause the user's lung capacity to increase as they become healthier through use of the device.

The device is pre-loaded with breathing exercises, i.e. lessons that teach particular patterns of breathing to improve health and energy levels. The measured flow rate can be used to identify whether the user is breathing in accordance with the lesson and if they are not, then the device can indicate corrections.

The memory unit 207 is partitioned into a number of sectors. The first is a database for storing data corresponding to breathing exercises. The memory unit 207 is capable of storing a number of data values in fields in the database for each parameter of the particular breathing exercise. A breathing exercise has the following parameters:
i) An identification code
ii) Lesson Name
iii) Cycle repetitions. These can gradually increase as the user progresses.
iv) Number of stages
v) Stage identifier
vi) Stage duration
vii) Stage sound field (sound effects, recording)
viii) Stage vibration state
ix) Stage valve setting
x) Corrective action. This field defines what corrective action is necessary if the detected flow rate is not as expected.

Parameters for specific breathing exercises are shown in the tables below.

Exercise ID: 1
Exercise name: “Box Breathing”
Cycle Repetitions: 8

| | | | | Vibration | Valve | |
|---|---|---|---|---|---|---|
| Stage | Duration | Sound | Audio | state | state | Corrective action |
| 1 | 8 | “Ching” | “Inhale | Slow | Open | Too slow: faster |
| | | | 1, 2, 3 | | | vibration, audio |
| | | | etc” | | | “faster inhale” |
| | | | | | | Too fast: slower |
| | | | | | | vibration, audio |
| | | | | | | “slow down”, valve |
| | | | | | | closes |
| | | | | | | proportionately. |
| 2 | 8 | 2 × | “Hold, | None | Closed | None |
| | | “Ching” | 1, 2, 3 | | | |
| | | | etc” | | | |
| 3 | 8 | 3 × | “Exhale, | Slow | Open | Too slow: faster |
| | | “Ching” | 1, 2, 3 | | | vibration, audio |
| | | | etc” | | | “faster exhale” |
| | | | | | | Too fast: slower |
| | | | | | | vibration, audio |
| | | | | | | “slow down”, valve |
| | | | | | | closes |
| | | | | | | proportionately. |
| 4 | 8 | 4 × | “Hold, | None | Closed | None |
| | | “Ching” | 1, 2, 3 | | | |
| | | | etc” | | | |

Exercise ID: 2
Exercise name: “Breath Observation”
Cycle Repetitions: 20

| | | Sound | | Vibration | Valve | |
|---|---|---|---|---|---|---|
| Stage | Time | effect | Audio | state | state | Corrective action |
| 1 | 6 | “Ching” | “Inhale 1, | Increasing | Open | See next row |
| | | | 2, 3 etc” | | | |

Breath rate too slow: faster vibration, audio “inhale faster”.
Breath rate too fast: slower vibration, audio “slow down”, valve closes proportionately.
Breath pause: audio “don't pause between breaths”.

| | | | | | | |
|---|---|---|---|---|---|---|
| 2 | 6 | 2 × | “Exhale, | Decreasing | Open | See next row |
| | | “Ching” | 1, 2, 3 | | | |
| | | | etc” | | | |

Breath rate too slow: faster vibration, audio “exhale faster”
Breath rate too fast: slower vibration, audio “slow down”, valve closes proportionately.
Breath pause: audio “don't pause between breaths”.

Exercise ID: 3
Exercise name: “20 Connected Breaths”
Stage Repetitions: Stage 1 × 4
Cycle Repetitions: 3
Exercise End: 3 × “Chime” repeated.

| | | Sound | | Vibration | Valve | |
|---|---|---|---|---|---|---|
| Stage | Time | effect | Audio | state | state | Corrective action |
| 1.1 | 2 | “Chime” | “Short | Fast | Open | None |
| | | | inhale” | | | |
| 1.2 | 2 | None | “Short | Fast | Open | None |
| | | | exhale” | | | |
| 2.1 | 8 | “Chime” × | “Long | Decreasing | Open | Breath rate too slow: |
| | | 2 | inhale, 1, | | | faster vibration, |
| | | | 2, 3 etc” | | | audio “inhale faster” |
| | | | | | | Breath rate too fast: |
| | | | | | | slower vibration, |
| | | | | | | audio “slow down”, |
| | | | | | | valve closes |
| | | | | | | proportionately. |
| 2.2 | 8 | None | “Long | Increasing | Open | Breath rate too slow: |
| | | | exhale, 1, | | | faster vibration, |
| | | | 2, 3 etc” | | | audio “exhale faster” |
| | | | | | | Breath rate too fast: |
| | | | | | | slower vibration, |
| | | | | | | audio “slow down”, |
| | | | | | | valve closes |
| | | | | | | proportionately. |

The memory unit 207 has a second sector for storing user specific variables, including:
i) Lung constant. This is referred to during the breathing exercises to determine the extent to which the user has completed the exercise.
i) Performance values, which is a record of the exercises completed and the past performance of a user.
ii) Exercise timetable, where the user can create and store an exercise program.

In use, there are a number of phases to the operation of the device.

### Set-Up Phase

The memory module 207 of the device is loaded with breathing exercise data. It can be loaded by the supplier of the device or by the user, via the data port 210 or the Bluetooth interface 216.

When the user first uses the device they are prompted to enter the calibration mode to determine their current lung constant.

### Exercises

A menu of available breathing exercises is presented on the display 215. The user selects an exercise that they wish to perform, either by touching the display or by pressing one of the control buttons 212. The selected breathing exercise is then initiated.

The CPU 206 accesses the memory unit 207 and runs a script according to the database entry for the selected breathing exercise. The operation of the device for delivering the “Box Breathing” exercise is described below.

The user initiates the lesson by pressing button 212. CPU 206 then interrogates the database entry for the selected breathing exercise and checks to see if there are any initial states that need to be set. In this example there are none. Then CPU 206 sets the states of the various units of the device as defined in Stage 1 for the Box Breathing exercise in the database, as follows:
Sound Effect: “Ching”
Sound recording: “Inhale for 1, 2, 3, 4, 5, 6, 7, 8 seconds” is played.
The vibration unit is set to slow vibration.
The valve is set to the open position.
The guide lights 1a to 7a are gradually illuminated over the course of 8 seconds.

The sensors 202 continuously measure the breath flow rate, Fᵣ. The CPU performs the calculation:

Fᵣ×tₑ

Where tₑ is the elapsed time from the start of the stage. The CPU monitors the result of the calculation and compares it to the lung constant C₁

When Fᵣ×tₑ=C₁/7, the first energy level light 1b is illuminated.

When Fᵣ×tₑ=2C₁/7, the second energy level light 2b is illuminated.

When Fᵣ×tₑ=3C₁/7, the third energy level light 3b is illuminated.

When Fᵣ×tₑ=4C₁/7, the fourth energy level light 4b is illuminated.

When Fᵣ×tₑ=5C₁/7, the fifth energy level light 5b is illuminated.

When Fᵣ×tₑ=6C₁/7, the sixth energy level light 6b is illuminated.

When Fᵣ×tₑ=7C₁/7, the seventh energy level light 7b is illuminated.

In this way the user can see how their breathing compares to the exercise as the guide lights 1a to 7a are illuminated with the ideal breathing pattern.

If the user inhales too slowly, i.e. the seventh energy level light is not illuminated by the time the Stage has finished, in this instance after 8 seconds, the corrective actions of increasing the vibration rate and playing a sound recording are performed to encourage the user to inhale faster next time.

If the user's inhale is too fast, i.e. the seventh energy level light is illuminated before the Stage has finished, i.e. before 8 seconds has elapsed, then the corrective action of decreasing the vibration, playing a recording and partially closing the valve is performed.

Detection of whether corrective action is required can be performed at any point during the Stage. As each one of the guide lights 1a to 7a is illuminated, a comparison is made with the users breath rate/lung constant ratio to determine whether they are ahead or behind in the exercise and the appropriate corrective action then implemented.

After the first Stage is complete, the CPU implements the second Stage of the exercise, which for Box Breathing involves the user holding their breath for 8 seconds. The CPU refers to the database and causes the sound effects and recordings. The valve closes so that the user is unable to breathe out. The guide lights 1a to 7a stay illuminated and the energy level lights remain at the level which the user reached before the end of the previous Stage.

The third Stage is then implemented, which requires the user to exhale for 8 seconds. The valve opens and the guide lights 1a to 7a extinguish over the course of the Stage, i.e. over a period of 8 seconds. The breath flow sensors detect the flow rate of the exhale and the CPU calculates when to extinguish the energy level lights. If the energy level is not aligned with the guide then the appropriate corrective action, as determined by the database, is implemented.

The fourth Stage is then initiated, which involves the user holding their breath for 8 seconds. The valve is shut and the energy level lights remain at the level which the user reached before the end of the stage.

The cycle is then repeated eight times.

At the end of the exercise, the user may access comparison data about how well they performed and how present performance compares to historical performance.

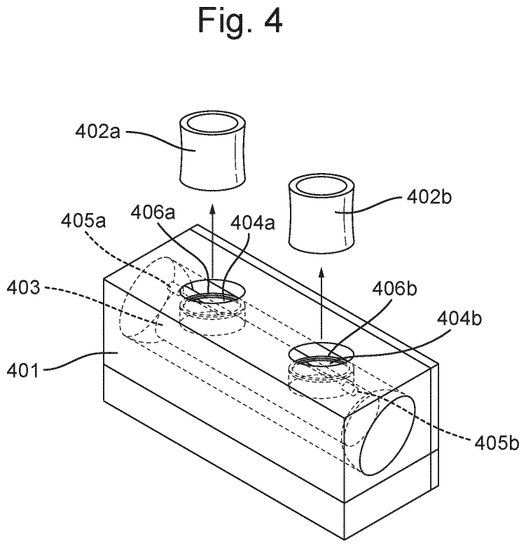

In another embodiment, a device is provided that is arranged to provide exercises for nasal breathing, as shown in FIG. 4.

The nasal breathing exercise device has a body 401. This can be held in place just below a user's nose. The device may have a strap arrangement to hold it in place (not shown). On the upper surface, a left nasal tube 402a, and a right nasal tube 402b are provided to engage with the nostrils of a user. These can be customised to fit a particular user or may be adjustable. The nasal tubes provide a conduit to a central channel 403, via openings 406a, b in the body 401. The central channel 403 is open at either end to provide vents to air. A filter may be provided over the outlets, which may be a mesh, gauze, fabric or electrostatic type. Valves 404a, 404b are provided in the openings 406a, b that are operable between an open and a closed position. The valves are operated by servos 405a, b. When valve 404a is open, it allows air to flow from the left nasal tube 402a to channel 403 and when it is closed, it prevents air from flowing. Similarly, when valve 404b is open, it allows air to flow from the left nasal tube 402b to the channel 403 and when it is closed, it prevents air from flowing. Flow sensors (not shown) are provided in the channel 403 to measure the flow of air. The sensors are able to measure the flow rate and direction of flow.

The device is battery powered and has a CPU, memory unit, controls and a screen. It may also have a Bluetooth enabled interface for communicating with other devices, such as a smartphone. The device may have a set of indicator lights, a first guide set of lights for displaying the breath exercise and a second energy level set of lights for displaying how full the user's lungs are with air, which also equates to energy level. The lights are located on the top surface of the device at either end so that the user can see them out of the corner of their eyes. A vibration unit is also provided that is arranged to vibrate at different frequencies to indicate the position in a breathing cycle.

The memory unit is arranged to store breathing exercises.

Parameters for a specific breathing exercise are shown in the table below.

Exercise ID: 4
Exercise name: “Alternate Nostril Breathing”
Cycle Repetitions: 5
End of cycle indicator: 3 sharp bursts of “Ching” with vibrational match.

| | | | | Vibration | Valve | |
|---|---|---|---|---|---|---|
| Stage | Time | Sound | Audio | state | state | Corrective action |
| 1 | 4 | “Ching” | “Fast | Increase | Left | Too slow: faster |
| | | | inhale | | nostril | vibration, audio |
| | | | left 1, 2, 3 | | valve | “faster inhale” |
| | | | etc” | | open, | Too fast: slower |
| | | | | | right | vibration, audio |
| | | | | | nostril | “slow down”, left |
| | | | | | valve | valve closes |
| | | | | | closed | proportionately. |
| 2 | 12 | 2 × | “Hold, 1, | None | Both | None |
| | | “Ching” | 2, 3 etc” | | valves | |
| | | | | | closed | |
| 3 | 8 | 3 × | “Slow | Decrease | Left | Too slow: faster |
| | | “Ching” | exhale | | nostril | vibration, audio |
| | | | right 1, 2, | | valve | “faster exhale” |
| | | | 3 etc” | | closed, | Too fast: slower |
| | | | | | right | vibration, audio |
| | | | | | nostril | “slow down”, right |
| | | | | | valve | valve closes |
| | | | | | open | proportionately. |
| 4 | 4 | 4 × | “Fast | Fast | Left | Too slow: faster |
| | | “Ching” | inhale | | nostril | vibration, audio |
| | | | right 1, 2, | | valve | “faster exhale” |
| | | | 3 etc” | | closed, | Too fast: slower |
| | | | | | right | vibration, audio |
| | | | | | nostril | “slow down”, right |
| | | | | | valve | valve closes |
| | | | | | open | proportionately. |
| 5 | 12 | 5 × | “Hold, 1, | None | Both | None |
| | | “Ching” | 2, 3 etc” | | valves | |
| | | | | | closed | |
| 6 | 8 | 6 × | “Slow | Decrease | Left | Too slow: faster |
| | | “Ching” | exhale | | nostril | vibration, audio |
| | | | left 1, 2, 3 | | valve | “faster exhale” |
| | | | etc” | | open, | Too fast: slower |
| | | | | | right | vibration, audio |
| | | | | | nostril | “slow down”, left |
| | | | | | valve | valve closes |
| | | | | | closed | proportionately. |

In operation, a suite of breathing exercises are loaded into the memory unit, either by the user or by the supplier of the device. The user then implements a calibration step, where they breathe all the way in through the left nostril. The left valve is opened and the right valve closed. The flow rate and duration are measured and used to calculate the lung constant. The same is repeated for the right nostril and the average of both nostrils taken.

An exercise is selected by the user and the CPU implements the exercise according to the database entry for the exercise.

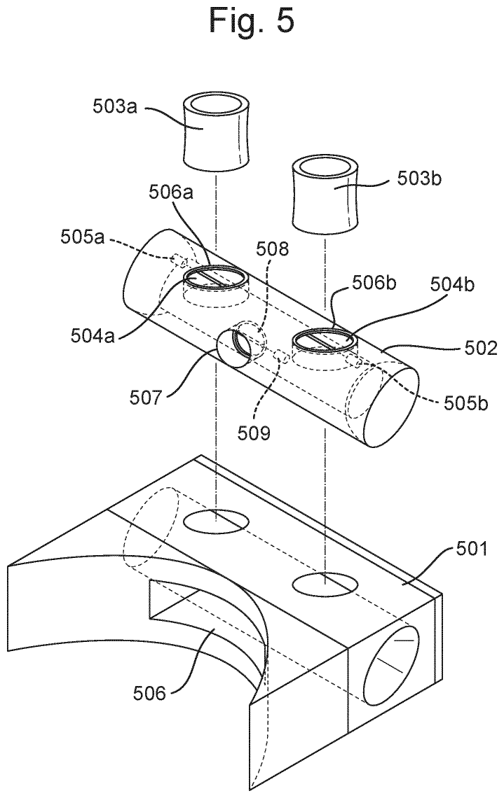

In an alternative embodiment, a device is provided that is a combination of a mouthpiece and nasal piece, as shown in FIG. 5. The body 501 includes a central channel piece 502, shown removed from the body in FIG. 5 for clarity, but in use it is located within the body 501. The channel piece 502 is open at both ends. It has two openings in the top surface for receiving a left nasal tube 503a and a right nasal tube 503b. The central channel has a front aperture 507. The front aperture 507 is closable by an electronic servo 509 and valve 508 assembly. The central channel has a left nostril valve 504a and a right nostril valve 504b to regulate the flow of air between the nasal tubes and the channel 502. The device includes a battery, CPU, sensors and display elements.

An example breathing exercise database format is shown below for a combination device:

Exercise ID: 5
Exercise name: “Energy Breath”
Cycle Repetitions: 3, with pulse phase increasing from 20 to 40 to 60.
End of cycle indicator: 3 sharp bursts of “Ching” with vibrational match.

| | | | | Vibration | Valve | |
|---|---|---|---|---|---|---|
| Stage | Time | Sound | Audio | state | state | Corrective action |
| 1 | 8 | “Chime” | “Inhale 1, | Increase | All valves | Too slow: faster |
| | | | 2, 3 etc” | | open | vibration, audio |
| | | | | | | “faster inhale” |
| | | | | | | Too fast: slower |
| | | | | | | vibration, audio |
| | | | | | | “slow down”, all |
| | | | | | | valves close |
| | | | | | | proportionately. |
| 2 | n/a | “Dong” | “Pulse, 1, | None | All valves | None |
| | | | 2, 3 to 20” | | open | |
| 3 | 8 | “Chime” | “Exhale 1, | Decrease | All valves | Too slow: faster |
| | | | 2, 3 etc” | | open | vibration, audio |
| | | | | | | “faster exhale” |
| | | | | | | Too fast: slower |
| | | | | | | vibration, audio |
| | | | | | | “slow down”, right |
| | | | | | | valve closes |
| | | | | | | proportionately. |
| 4 | 4 | “Chime” | “Fast | Fast | All valves | Too slow: faster |
| | | | inhale 1, | | open | vibration, audio |
| | | | 2, 3, 4” | | | “faster exhale” |
| | | | | | | Too fast: slower |
| | | | | | | vibration, audio |
| | | | | | | “slow down”, right |
| | | | | | | valve closes |
| | | | | | | proportionately. |
| 5 | 30 | “Chime” | “Hold, 1, | None | All valves | None |
| | | | 2, 3 etc” | | closed | |
| 6 | 8 | “Chime” | “Slow | Decrease | All valves | Too slow: faster |
| | | | exhale | | open. | vibration, audio |
| | | | left 1, 2, 3 | | | “faster exhale” |
| | | | etc” | | | Too fast: slower |
| | | | | | | vibration, audio |
| | | | | | | “slow down”, left |
| | | | | | | valve closes |
| | | | | | | proportionately. |

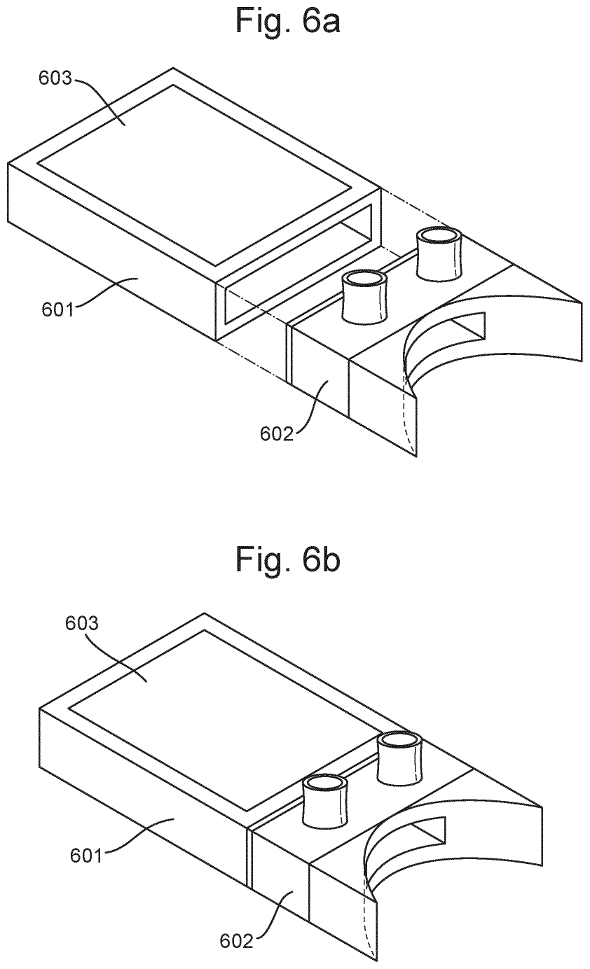

The nasal device of FIG. 4 and the combination mouth and nasal device of FIG. 5 are standalone units. In a further embodiment, the intelligence of the device, including CPU, sensors, battery and displays may be provided in a separate base unit 601 as shown in FIG. 6a. The mouthpiece and nasal piece including valves provided in a separate unit that can be attached to the base unit 601, as shown in FIG. 6b. This allows a selection of either mouth piece, nasal piece or combination mouth and nasal piece to be selected by the user.

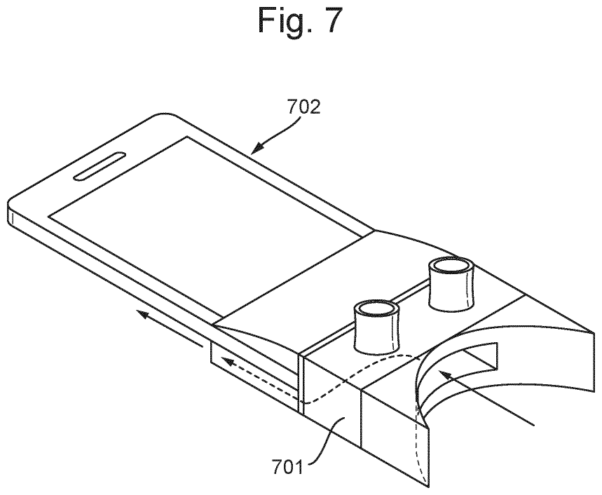

In a further embodiment shown in FIG. 7, the mouthpiece, nasal piece or combination of mouthpiece and nasal piece 701 may be provided with minimal electronic circuitry, such as valves, valve servos and sensors, and a Bluetooth link. A separate smartphone 702 may be loaded with an application that supports the device and provides data analysis and feedback.

In another embodiment, a breath guide device is provided that does not measure breathflow but simply opens and closes the valves according to the breathing exercise stored in the memory.

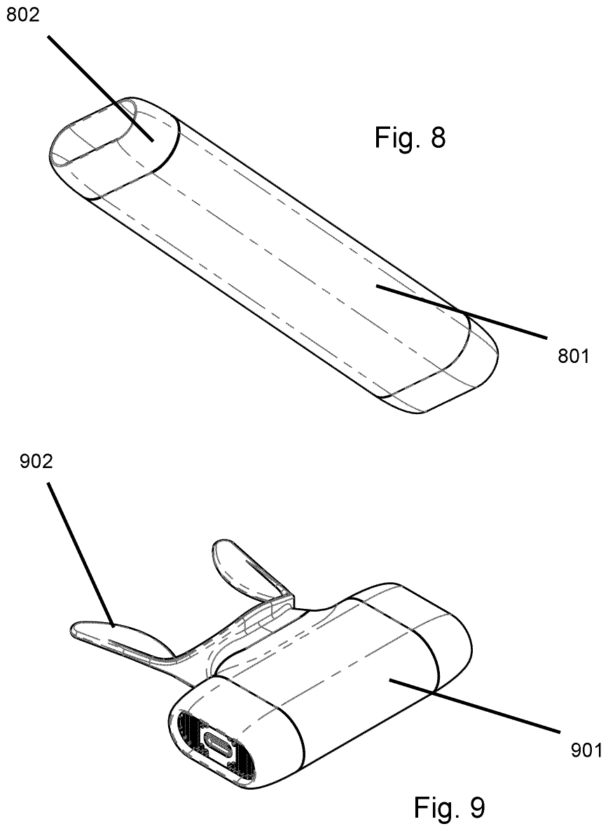

This device could simply be a mouthpiece 801 having a housing 802 and an electronically operated valve, controlled in accordance with a set of breathing exercises stored in a memory, as shown in FIG. 8. Alternatively, it could simply be a nose-piece having two channels that can be engaged with a user's nostrils, where each nostril channel has an electronically operated valve, each controlled in accordance with a set of breathing exercises stored in a memory. The device could be made as a “hands-free” assembly, as shown in FIG. 9, where a body 901 has a fitting 902 for a user to put in their mouth and grip with their teeth.

1-25. (canceled)

26. A breath guide device having;
an airway,
breathflow guiding equipment comprising an electronically operated valve for regulating the flow of breath through the airway, the guiding equipment further comprising haptic feedback apparatus including a vibration unit,
the device further having a control unit capable of communicating with a memory unit, the memory unit capable of storing data representing an ideal breath flow rate,
the device further including a breathflow measurement unit in the airway arranged to measure the breath flow rate of a user
and wherein the control unit is further arranged to compare the data representing the ideal breath flow rate with the data representing the measured breath flow rate of the user, and to control the valve and the vibration unit in dependence on the result of the comparison.

27. The breath guide device of claim 26, including a mouthpiece suitable for placement at a user's mouth.

28. The breath guide device of claim 26, including a nasal piece comprising two nasal conduits for engagement with a user's nostrils and wherein the breathflow guiding equipment includes an electronically operated valve in each of the two nasal conduits for regulating the flow of breath through the airway.

29. The breath guide device of claim 26, wherein the data capable of being held in the memory is data defining a breathing exercise.

30. The breath guide device of claim 29, further including a mouthpiece suitable for placement at a user's mouth, wherein the control unit is arranged to close the valve if the user's breath flow rate is detected as being higher than the preset breath rate.

31. The breath guide device of claim 29, including a nasal piece comprising two nasal conduits for engagement with a user's nostrils and wherein the breathflow guiding equipment includes an electronically operated valve in each of the two nasal conduits for regulating the flow of breath through the airway, wherein the control unit is arranged to close one of the valves if the user's breath flow rate is detected as being higher than the preset breath rate.

32. The breath guide device of claim 29, wherein the breathflow measurement unit includes one or more IR sensors.

33. The breath guide device of claim 29, wherein the breathflow measurement unit includes one or more LiDAR sensors.

34. The breath guide device of claim 29, wherein the breathflow measurement unit includes one or more turbine sensors.

35. The breath guide device of claim 26, wherein the breathflow guiding equipment includes a plurality of visual indicators.

36. The breath guide device of claim 26, wherein the breathflow guiding equipment includes an audible indicator.

37. The breath guide device of claim 36, wherein the audible indicator is capable of reproducing pre-recorded spoken words.

38. The breath guide device of claim 36, wherein the data capable of being held in the memory is data defining a breathing exercise, wherein the audible indicator is arranged to operate if the user's breath flow rate is detected as being higher or lower than the expected breath flow rate.

39. The breath guide device of claim 36, wherein the breathflow guiding equipment includes a vibrational indicator.

40. The breath guide device of claim 39, wherein the data capable of being held in the memory is data defining a breathing exercise, wherein the vibrational indicator is arranged to operate if the user's breath flow rate is detected as being higher or lower than the expected breath flow rate.

41. The breath guide device of claim 26, wherein the memory unit is located within the device.

42. The breath guide device of claim 26, wherein the memory unit is remote from the device.

43. A method of operating a breath guide device including the steps of
reading data representing a breathing exercise held in a memory unit,
controlling breathflow guiding equipment located in the device in accordance with the data representing a breathing exercise.

44. The method of claim 43, wherein the breathflow guiding equipment is at least one valve and the method involves opening and closing the at least one valve in accordance with the data representing a breathing exercise.

45. The method of claim 43, wherein the breathflow guiding equipment is a plurality of indicator lights and the method involves illuminating the indicator lights in accordance with the data representing a breathing exercise.
